(19) United States
(12) Patent Application Publication
(10) Pub. No.: US 2005/0197372 A1
(43) Pub. Date: Sep. 8, 2005

(54) Use of valsartan or its metabolite to inhibit platelet aggregation

(76) Inventors: Alex Malinin, Cockeysville, MD (US); Victor Serebruany, West Friendship, MD (US); Randy Lee Webb, Flemington, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/514,299

(22) PCT Filed: May 13, 2003
(86) PCT No.: PCT/EP03/04997

§ 371 Date: Nov. 12, 2004

(30) Foreign Application Priority Data
May 14, 2002 (US) 60380373
Jul. 11, 2002 (US) 60395014

(51) Int. Cl. A61K 31/4184

(52) U.S. Cl. 514/381

(57) ABSTRACT

The invention relates to a method of inhibiting platelet aggregation comprising administering a therapeutically effective amount of an ARB or its metabolite, especially Valsartan or its metabolite valeryl 4-hydroxy valsartan. Conditions to be treated by inhibition of platelet aggregation include acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

### BACKGROUND OF THE INVENTION

Valsartan selectively blocks the binding of angiotensin II to the A_{T1} receptor causing vasodilatation, and diminishes aldosterone secretion. Recent clinical studies revealed additional benefits of Valsartan in a cohort of patients after acute vascular events. Considering that platelet activation plays a key role in the pathogenesis of coronary and cerebrovascular occlusion, and that A_{T1} receptors are present on the platelet surface the in vitro effects of Valsartan and its major liver metabolite, valeryl 4-hydroxy valsartan on platelets in subjects with multiple risk factors for vascular disease was assessed.

### BRIEF DESCRIPTION OF THE DRAWINGS

The following drawings depict certain embodiments of the invention. They are illustrative only and do not limit the invention otherwise disclosed herein.

FIG. 1A-C. Actual scanned platelet aggregation curves from a study patient at baseline (A), and after incubation with 100 μM of valsartan (B), and 1 μM of VALERYL 4-HYDROXY VALSARTAN (C). High concentrations of valsartan significantly inhibit ADP-induced aggregation (B), with no effect on epinephrine-induced aggregability, while the metabolite blocks epinephrine-induced aggregation (C) in the therapeutic concentration range.

### SUMMARY OF THE INVENTION

In one aspect the present invention relates to a method of inhibiting platelet aggregation comprising administering a therapeutically effective amount of an angiotensin II receptor blocker (“ARB”), preferably valsartan, or pharmaceutically acceptable salts thereof, optionally in the presence of a pharmaceutically acceptable carrier, to a patient in need thereof.

In another embodiment the present invention relates to a method of inhibiting platelet aggregation comprising administering a therapeutically effective amount of a metabolite of an ARB, especially the metabolite of Valsartan, valeryl 4-hydroxy valsartan, optionally in the presence of a pharmaceutically acceptable carrier, to a patient in need thereof.

In another aspect of the present invention there is provided a method of treating conditions associated with platelet aggregation comprising administering an ARB or a metabolite of an ARB to a patient in need thereof. Conditions associated with platelet aggregation include acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

Another aspect of the present invention relates to pharmaceutical compositions comprising an ARB or a metabolite of an ARB and a pharmaceutically acceptable carrier.

### DETAILED DESCRIPTION OF THE PREFERRED EMBODIMENTS

AT₁-receptor antagonists (also called angiotensin II receptor antagonists) are understood to be those active ingredients which bind to the AT₁-receptor subtype of angiotensin II receptor but do not result in activation of the receptor. As a consequence of the inhibition of the AT₁ receptor, these antagonists can, for example, be employed as to prevent platelet aggregation and treat conditions associated therewith.

The class of AT₁ receptor antagonists comprises compounds having differing structural features, essentially preferred are the non-peptidic ones. The ARBs within the scope of the present invention include valsartan, which is the AT 1 receptor antagonist (S)-N-(1-carboxy-2-methyl-prop-1-yl)-N-pentanoyl-N-[2;(1H-tetrazol-5-yl)biphenyl-4-yl-methyl]amine of formula (I) 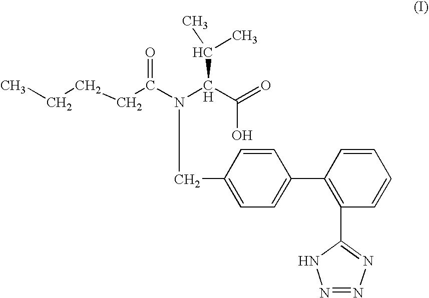
and is disclosed in EP 0443983 A and U.S. Pat. No. 5,399,578, the disclosures of which are incorporated herein in their entirety as if set forth herein. Other ARB compounds include, but are not limited to, losartan, candesartan, eprosartan, irbesartan, saprisartan, tasosartan, telmisartan, olmesartan, zolarsartan (1-[[3-bromo-2-[2-(1H-tetrazol-5-yl)phenyl]-5-benzo-furanyl]methyl]-2-butyl-4-chloro-1H-imidazole-5-carboxylic acid, and 3-(3-Bromo-2-[2-(1H-tetrazol-5-yl)-phenyl]-bezofuran-5-yl methyl)₂-butyl-5-chloro-3H-imidazole-4-carboxylic acid, methyl 2-[[4-butyl-2-methyl-6-oxo-5-[[2′-(1H-tetrazol-5-yl)[1,1′-biphenyl]4-yl]methyl]-1-(6H)-pyrimidinyl]methyl]-3-thiophenecarboxylate also known as LR-B/081 and Methyl 2-[[4-butyl-2-methyl-6-oxo-5-[[2′-(1H-tetrazol-5-yl)[1,1′-biphenyl]-4-yl]methyl]-1-(6H)pyrimidinyl]methyl]-3-thiophenecarboxylate also known as 3k, LR-B/081 which has the introduction of a (carboxyheteroaryl)methyl moiety at the 3-position (Lusofarmaco), 2,7-diethyl-5-[[2′-(1H-tetrazole-5-yl)biphenylyl-4-yl]methyl]-5H-pyrazolo[1,5-b][1,2,4]-triazole potassium salt also known as YM 358 (Yamanouchi) disclosed in Biol Pharm Bull 2000 February; 23(2): 174-81, L-158,809 disclosed in Thromb Res 2002 Mar. 15; 105(6): 531-6, KT3 671 disclosed in J Cardiovasc Pharmacol 1995 January; 25(1): 22-9, TA 606 disclosed in J Cardiovasc Pharmacol 1998 April; 31(4): 568-75, TH 142177 disclosed in Fundam Clin Pharmacol 1997; 11(5): 395-401, UP 269-6 disclosed in Br J Pharmacol 1997 February; 120(3): 488-94, the compound with the designation E-1477 of the following formula 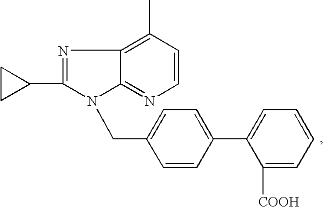
the compound with the designation SC-52458 of the following formula 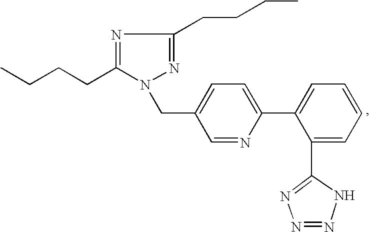
and the compound with the designation the compound ZD-8731 of the following formula 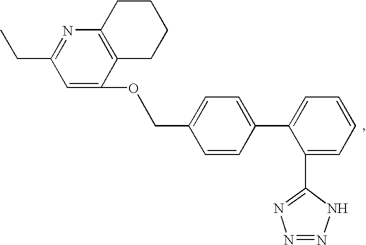
or, in each case, a pharmaceutically acceptable salt thereof.

It has also been surprisingly found that metabolites of ARBs also significantly reduce platelet aggregation. Metabolites of ARBs include
metabolite of losartan, which is, 2-n-Butyl-4-chloro-1-[(2′-(1H-tetrazol-5-yl)-biphenyl-4-yl)methyl]imidazole-5-carboxylic acid hydrochloride also called EXP-3174 as disclosed in J Pharmacol Exp Ther 1990 October; 255(1): 211-7 and the metabolite EXP-3174 (II) disclosed in J Chromatogr 1992 Jan. 17; 573(2): 295-301;
metabolites of irbesartan, which are 1) a tetrazole N2-beta-glucuronide conjugate of irbesartan, (2) a monohydroxylated metabolite resulting from omega-1 oxidation of the butyl side chain, (3, 4) two different monohydroxylated metabolites resulting from oxidation of the spirocyclopentane ring, (5) a diol resulting from omega-1 oxidation of the butyl side chain and oxidation of the spirocyclopentane ring, (6) a keto metabolite resulting from further oxidation of the omega-1 monohydroxy metabolite, (7) a keto-alcohol resulting from further oxidation of the omega-1 hydroxyl of the diol, and (8) a carboxylic acid metabolite resulting from oxidation of the terminal methyl group of the butyl side chain disclosed in Drug Metab Dispos 1998 May; 26(5): 408-17; metabolite of candesartan cilexetil, which is candesartan (2-ethoxy-1-[[2′-(1H-tetrazol-5-yl)biphenyl-4-yl]methyl]-1H-benzimidazole-7-carboxylic acid), also known as CV 11974 and CV-15959 disclosed in Clin Pharmacokinet 2002; 41(1): 7-17, J Chromatogr B Biomed Sci Appl 1999 Aug. 20; 731(2): 411-7, and J Hum Hypertens 1997 September; 11 Suppl 2: S19-25;
metabolite of telmisartan, which is telmisartan 1-O-acylglucuronide disclosed in Drug Metab Dispos 1999 October; 27(10): 1143-9;
metabolite of olmesartan medoxomil, which is olmesartan or CS866, as disclosed in J Hypertens Suppl 2001 June; 19 Suppl 1: S21-32;
metabolite of eprosartan, which is glucuronide disclosed in Pharmacotherapy 1999 April; 19(4 Pt 2): 73S-78S;
metabolite of tasosartan, which is enoltasosartan disclosed in J Pharmacol Exp Ther 2000 November; 295(2): 649-54 and five other metabolites disclosed in J. Med. Chem. 1998, Oct. 22, 41(22), 4251-60;
metabolite of zolarsartan, which is glucuronic acid conjugates and five related to biotransformation products, three hydroxylated on the aliphatic side chain, one further oxidized to a ketone and one hydroxylated on the phenyl ring disclosed in J Pharm Biomed Anal 1994 September; 12(9): 1181-7 and J Pharm Biomed Anal 1994 September; 12(9): 1181-7;
metabolite of ZD-8731;
metabolite of (5-[(3,5-dibutyl-1H-1,2,4-triazol-1-yl)methyl]-2-[2-(1H-tetrazol-5-ylphenyl)]pyridine also known as SC-52458 disclosed in J Cardiovasc Pharmacol 1993 October; 22(4): 617-25;
and metabolites of the following ARBs, ZD-8731 (Zeneca), methyl 2-[[4-butyl-2-methyl-6-oxo-5-[[2′-(1H-tetrazol-5-yl)[1,1′-biphenyl]-4-yl]methyl]-1-(6H)pyrimidinyl]methyl]-3-thiophenecarboxylate also known as LR-B/081 and Methyl 2-[[4-butyl-2-methyl-6-oxo-5-[[2′-(1H-tetrazol-5-yl)[1,1′-biphenyl]-4-yl]methyl]-1-(6H)-pyrimidinyl]methyl]-3-thiophenecarboxylate also known as 3k, LR-B/081 which has the introduction of a (carboxyheteroaryl)methyl moiety at the 3-position (Lusofarmaco), 2,7-diethyl-5-[[2′-(1H-tetrazole-5-yl)biphenyl-4-yl]methyl]-5H-pyrazolo[1,5-b][1,2,4]-triazole potassium salt also known as YM 358 (Yamanouchi) disclosed in Biol Pharm Bull 2000 February; 23(2): 174-81, L-158,809 disclosed in Thromb Res 2002 Mar. 15; 105(6): 531-6, KT3 671 disclosed in J Cardiovasc Pharmacol 1995 January; 25(1): 22-9, TA 606 disclosed in J Cardiovasc Pharmacol 1998 April; 31(4): 568-75, TH 142177 disclosed in Fundam Clin Pharmacol 1997; 11(5): 395-401 and UP 269-6 disclosed in Br J Pharmacol 1997 February; 120(3): 488-94.

Preferred is the metabolite of Valsartan which is valeryl 4-hydroxy valsartan having the formula 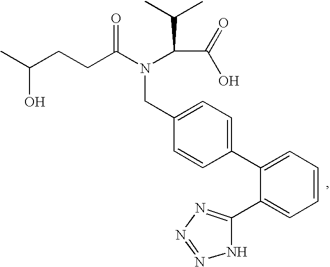
disclosed in Waldmeier F., et al., Xenobiotica, 1997, Vol. 27, No. 1, 59-71, hereby incorporated by reference in its entirety as if set forth in full herein.
ARBs and their metabolites may be referred to herein as “the compounds of the invention”. The compounds of the invention depending on the nature of the substituents, may possess one or more asymmetric centers. The resulting diastereoisomers, enantiomers and geometric isomers are encompassed by the instant invention.

Depending on the choice of starting materials and methods, the compounds may be in the form of one of the possible isomers or mixtures thereof, for example, as substantially pure geometric (cis or trans) isomers, optical isomers (antipodes), racemates, or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, for example by chromatography and/or fractional crystallization.

Any resulting racemates of final products or intermediates can be resolved into the optical antipodes by known methods, e.g. by separation of the diastereoisomeric salts thereof, obtained with an optically active acid or base, and liberating the optically active acidic or basic compound. The carboxylic acid intermediates can thus be resolved into their optical antipodes e.g. by fractional crystallization of D or L-(alpha-methylbenzylamine, cinchonidine, cinchonine, quinine, quinidine, ephedrine, dehydroabietylamine, brucine or strychnine)-salts. Racemic products can also be resolved by chiral chromatography, e.g. high pressure liquid chromatography using a chiral adsorbent.

Finally, compounds of the invention are either obtained in the free form, or as a salt thereof if salt forming groups are present.

Acidic compounds of the invention may be converted into salts with pharmaceutically acceptable bases, e.g. an aqueous alkali metal hydroxide, advantageously in the presence of an ethereal or alcoholic solvent, such as a lower alkanol. From the solutions of the latter, the salts may be precipitated with ethers, e.g. diethyl ether. Resulting salts may be converted into the free compounds by treatment with acids. These or other salts can also be used for purification of the compounds obtained.

Compounds of the invention having basic groups can be converted into add addition salts, especially pharmaceutically acceptable salts. These are formed, for example, with inorganic acids, such as mineral adds, for example sulfuric acid, a phosphoric or hydrohalic add, or with organic carboxylic acids, such as (C₁-C₄)-alkanecarboxylic acids which, for example, are unsubstituted or substituted by halogen, for example acetic add, such as saturated or unsaturated dicarboxylic acids, for example oxalic, succinic, maleic or fumaric add, such as hydroxy-carboxylic acids, for example glycolic, lactic, malic, tartaric or citric acid, such as amino acids, for example aspartic or glutamic acid, or with organic sulfonic acids, such as (C₁-C₄)-alkyl-sulfonic acids (for example methanesulfonic acid) or arylsulfonic acids which are unsubstituted or substituted (for example by halogen). Preferred are salts formed with hydrochloric acid, methanesulfonic acid and maleic acid.

In view of the close relationship between the free compounds and the compounds in the form of their salts, whenever a compound is referred to in this context, a corresponding salt is also intended, provided such is possible or appropriate under the circumstances.

The compounds, including their salts, can also be obtained in the form of their hydrates, or include other solvents used for their crystallization.

Another aspect of the invention includes pharmaceutical compositions comprising a therapeutically effective amount of the compound of the invention and a pharmaceutically acceptable carrier. The pharmaceutical compositions according to the invention can be prepared in a manner known per se and are those suitable for enteral, such as oral or rectal, and parenteral administration to mammals (warm-blooded animals), including man, comprising a therapeutically effective amount of the pharmacologically active compound, alone or in combination with one or more pharmaceutically acceptable carriers, especially suitable for enteral or parenteral application. Typical oral formulations include tablets, capsules, syrups, elixirs and suspensions. Typical injectable formulations include solutions and suspensions. The pharmaceutical compositions may be employed for the treatment of conditions mediated by platelet aggregation, in particular, acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

The typical pharmaceutically acceptable carriers for use in the formulations described above are exemplified by: sugars such as lactose, sucrose, mannitol and sorbitol; starches such as cornstarch, tapioca starch and potato starch; cellulose and derivatives such as sodium carboxymethyl cellulose, ethyl cellulose and methyl cellulose; calcium phosphates such as dicalcium phosphate and tricalcium phosphate; sodium sulfate; calcium sulfate; polyvinylpyrrolidone; polyvinyl alcohol; stearic acid; alkaline earth metal stearates such as magnesium stearate and calcium stearate; stearic acid; vegetable oils such as peanut oil, cottonseed oil, sesame oil, olive oil and corn oil; non-ionic, cationic and anionic surfactants; ethylene glycol polymers; betacyclodextrin; fatty alcohols; and hydrolyzed cereal solids, as well as other non-toxic compatible fillers, binders, disintegrants, buffers, preservatives, antioxidants, lubricants, flavoring agents, and the like commonly used in pharmaceutical formulations.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compounds. Pharmaceutical preparations for enteral or parenteral administration are, for example, in unit dose forms, such as coated tablets, tablets, capsules or suppositories and also ampoules. These are prepared in a manner which is known per se, for example using conventional mixing, granulation, coating, solubulizing or lyophilizing processes. Thus, pharmaceutical preparations for oral use can be obtained by combining the active compounds with solid excipients, if desired granulating a mixture which has been obtained, and, if required or necessary, processing the mixture or granulate into tablets or coated tablet cores after having added suitable auxiliary substances.

ARBs, especially valsartan, and metabolites of ARBs, especially valeryl 4-hydroxy valsartan, may be combined with other therapeutic agents, e.g., each at an effective therapeutic dose as reported in the art. Such therapeutic agents include heparin, warfarin, t-PA, urokinase, streptokinase, aspirin, ticlopidine, clopidogrel, abciximab, eptifibatide and tirofiban, anti-hypertensive agents and anti-diabetics.

The dosage of the active compound can depend on a variety of factors, such as mode of administration, homeothermic species, age and/or individual condition.

Preferred dosages for the active ingredients according to the present invention are therapeutically effective dosages, especially those which are commercially available for valsartan.

Normally, in the case of oral administration of the compounds of the present invention, an approximate daily dose of from about 0.1 mg to about 360 mg is to be estimated e.g. for a patient of approximately 75 kg in weight.

Valsartan is supplied in the form of suitable dosage unit form, for example, a capsule or tablet, and comprising a therapeutically effective amount, e.g. from about 20 to about 320 mg, of valsartan which may be applied to patients. The application of the active ingredient may occur up to three times a day, starting e.g. with a daily dose of 20 mg or 40 mg of valsartan, increasing to 80 mg daily and further to 160 mg daily up to 320 mg daily. Preferably, valsartan is applied once a day or twice a day to patients with a dose of 80 mg or 160 mg, respectively, each. Corresponding doses may be taken, for example, in the morning, at mid-day or in the evening.

In case of valerly 4-hydroxy valsartan, preferred dosage unit forms are, for example, tablets or capsules comprising e.g. for a mammal of about 50 to 70 kg from about 1 mg to about 1000 mg, advantageously from about 5 mg to about 500 mg, even more advantageously from about 20 mg to about 320 mg, administered once a day.

The above doses encompass a therapeutically effective amount of the active ingredients of the present invention.

It has surprisingly been found that both ARBs, particularly valsartan and the metabolites of ARBs, particularly valeryl 4-hydroxy valsartan exhibit significant in vitro inhibition of human platelets.

The compounds of the present invention inhibit platelet aggregation, and thus may be employed for the treatment of conditions mediated by platelet aggregation, in particular, acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

The invention furthermore also relates to a compound according to the invention for use in the prevention of, delay of progression of, treatment of a disease or condition mediated by platelet aggregation, in particular, acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

The invention furthermore also relates to the use of a compound according to the invention for the manufacture of a medicament for the prevention, delay of progression or treatment of a disease and disorder mediated by platelet aggregation, in particular, acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

The above-cited properties have been demonstrated by the following method: Blood samples for platelet aggregation, flow cytometric studies, and cartridge-based platelet assay analyzers were obtained from 20 volunteers with known vascular risk factors. Study participants were excluded if they had a history of bleeding diathesis, history of stroke, major surgery or significant trauma in the past six months, and hypertension of more than 200/110 mm. None of them received aspirin or any other anti-platelet agents. All subjects underwent blood sampling after at least 30 minutes of rest and 2 or more hours of fasting. Blood was drawn between 8 and 10 a.m. in order to avoid any diurnal influence and sampled from an antecubital vein using a 21-gauge butterfly needle containing 3.8% sodium citrate (1:9 volume) after having discarded the first 1.5 ml of free running blood. Eleven Vacutainer tubes (4.5 ml) were collected for a total of 49 ml of whole blood-citrate mixture from each study participant. One tube was kept as an internal control and was incubated with buffer solution. Five tubes were incubated with valsartan for 60 minutes at 37° C. in order to achieve the final concentrations of the compound at 10 nM, 100 nM, 1 μM, 10 μM, and 100 μM. The remaining five tubes were similarly incubated with increasing amounts of valeryl 4-hydroxy valsartan to achieve concentrations of 10 nM, 100 nM, 1 μM, 10 μM, and 100 μM. The concentrations of valsartan and valeryl 4-hydroxy valsartan ranged from subtherapeutic to markedly supra-therapeutic plasma levels observed in patients undergoing valsartan therapy. Valsartan peak plasma concentration can reach as high as 7.5 μM after a 160 mg intake, while valeryl 4-hydroxy valsartan in plasma represents only 10% of valsartan levels. Fresh solutions of valsartan and valeryl 4-hydroxy valsartan were prepared ex tempore on the same morning the platelet studies were performed. To avoid possible observer bias, blood samples were coded and blinded. Sampling procedures, and platelet studies were performed by individuals unaware of the protocol.

Platelet Aggregation

A. Platelet-Rich Plasma

The citrate and whole blood mixture were centrifuged at 1200 g for 5 minutes in order to obtain platelet-rich plasma (PRP) which was kept at room temperature for use within 1 hour. Platelet counts were determined for each PRP sample with a Coulter Counter ZM (Coulter Co., Hialeah, Fla.). Platelet numbers were adjusted to 3.50×10 ⁸/ml with homologous platelet-poor plasma. Platelet aggregation (PA) was induced by 5 μM ADP, and 5 μM epinephrine. All agonists were obtained from Chronolog Corporation (Havertown, Pa.). Aggregation studies were performed using a 4 channel Chronolog Lumi-Aggregometer (model 560-Ca). Aggregation was expressed as the maximum percentage of light transmittance change (% max) from the baseline at the end of the recording time, using platelet-poor plasma as a reference. Aggregation curves were recorded for 6 minutes and analyzed according to internationally established standards. Ruggeri Z M, Semin Hemat 1994; 31: 229-239.

B. Whole Blood

The methods are described in detail in Abbate R, et al., Amer J Clin Pathol 1986; 86: 91-96. Briefly, the whole blood citrate mixture was diluted 1:1 with 0.5 ml TBS, then swirled gently. The cuvette with the stirring bar was placed in the incubation well and allowed to warm to 37° C. for 5 minutes. Then the sample was transferred to the assay well. An electrode was placed in the sample cuvette. Platelet aggregation was stimulated with 1 μg/ml collagen. Platelet aggregation studies were performed using a Chrono-Log Whole Blood Aggregometer using Aggrolink” software. Platelet aggregability was expressed as the change in electrical impedance and is expressed in ohms.

Whole Blood Flow Cytometry

The expression of platelet receptors was determined by using the following monoclonal antibodies: CD31 (platelet endothelial cell adhesion molecule (PECAM-1), CD41 (glycoprotein [GP] IIb/IIIa, (IIb (3), CD42b (GP Ib), CD 51/CD61 ((v (3, or vitronectin receptor), CD62p (P-selectin), CD107a (lysosome associated membrane protein-1; LAMP-1), CD 107b (LAMP-2), CD151 (platelet/endothelial tetraspan antigen-3; PETA-3), and PAC-1 for fibrinogen-platelet (PharMingen, San Diego, Calif.). Platelet-leukocyte interactions were assessed by using dual antibodies for a pan-platelet marker (CD 151), together with CD14, a monocyte/macrophage marker. The blood-citrate mixture (50 μl) was diluted with 450 μl Tris buffered saline (TBS) (10 mmol/L Tris, 0.15 mol/L sodium chloride) and mixed by gently inverting an Eppendorf tube 2 times. Five μl of the corresponding antibodies were then added to each solution and the samples were incubated for 30 minutes. After incubation, 400 μl of 2% buffered paraformaldehyde was added for fixation. The samples were analyzed by a Becton Dickinson FACScan flow cytometer set up to measure fluorescent light scatter, as described in Gurbel P A, et al., J Am Coll Cardiol. 1998; 31: 1466-1473. The data were collected in list mode files and then analyzed. P selectin was expressed as percent positive cells as described in Gurbel P A, et al, Am Heart J 2000; 139: 320-328. Other antigens were expressed as log mean fluorescence intensity.

Cartridge-Based Platelet Analyzers

The platelet function analyzer (PFA-100′, Dade Behring, Deerfield, Ill.) is a device that simulates changes in primary hemostasis after injury to a small vessel under flow conditions. Kundu S K, et al., Semin Thromb Hemost 1995; 21(Suppl 2): 106-112. The time required to obtain occlusion of the aperture was digitally recorded as a measure of shear-induced platelet aggregation. Closure time determinations were performed in duplicate.

A rapid platelet-function assay cartridge test (RPFA-ASA, Ultegra® Accumetrics, Inc., San Diego, Calif., USA), using polystyrene beads coated cartridges with lyophilized human fibrinogen-coated microparticles, and propyl gallat served as an agonist. The whole blood citrate mixture was added to the cartridge, and agglutination between platelets and coated beads was recorded. The data mirrored turbidometric platelet aggregation and reflected the degree of platelet prostaglandin blockade. Smith J W, et al., Circulation 1999; 99: 620-625. Ultegra® assays were performed in duplicate. An electronic quality control test was performed on each instrument every day of use prior to performing any subject samples.

Statistical Analysis

All comparisons were calculated by Student's t-test to identify specific differences in platelet aggregation, results of Ultegra®, Dade-PFA 100™, and receptor expression between baseline and post valsartan/valeryl 4-hydroxy valsartan incubation. The Mann-Whitney U test was used to analyze non-parametric data. Normally distributed data were expressed as mean±SE, and skewed data as median (range). Probability values of p<0.05 were regarded as statistically significant. Linear regression analysis was applied to normally distributed data for all study participants by using the SPSS v9.0 program (SPSS Inc. Chicago, Ill.) for statistical analysis.

Platelet Aggregation in Platelet-Rich Plasma

Preincubation with escalating doses of valsartan resulted in inhibition of ADP-induced platelet aggregation only at a concentration of 100 μM which exceeds the therapeutic level, and did not affect the epinephrine-induced platelet aggregation. In contrast, incubation with valeryl 4-hydroxy valsartan inhibited epinephrine-induced aggregation in concentration 1 μM and 10 μM, and did not have any effect on ADP-induced aggregation. Representative data for 5 μM ADP and 5 μM epinephrine-stimulated aggregation are shown at Table 1.

TABLE 1
PRP platelet aggregation

| | | | Valeryl 4-hydroxy valsartan | | |
|---|---|---|---|---|---|
| | | | (valeryl 4-hydroxy | | p-value |
| Variable | Valsartan (V) | | valsartan) | | V vs. |
| Drug | | p-value | | p-value | valeryl 4- |
| concen- | | vs. | | vs. | hydroxy |
| tration | | baseline | | baseline | valsartan |

Platelet rich plasma aggregation induced by 5 μM ADP (%)

| Baseline | | 75 ± 8 | | 75 ± 8 | | |
|---|---|---|---|---|---|---|
| 10 | nM | 74 ± 6 | NS | 75 ± 6 | NS | NS |
| 100 | nM | 75 ± 8 | NS | 76 ± 8 | NS | NS |
| 1 | μM | 76 ± 7 | NS | 77 ± 9 | NS | NS |
| 10 | μM | 75 ± 8 | NS | 76 ± 8 | NS | NS |
| 100 | μM | 55 ± 12 | 0.0002 | 77 ± 9 | NS | 0.001 |

Platelet rich plasma platelet aggregation induced by
5 μM epinephrine (%)

| Baseline | | 78 ± 10 | | 78 ± 10 | | |
|---|---|---|---|---|---|---|
| 10 | nM | 77 ± 11 | NS | 77 ± 10 | NS | NS |
| 100 | nM | 76 ± 10 | NS | 76 ± 9 | NS | NS |
| 1 | μM | 78 ± 6 | NS | 54 ± 11 | 0.0001 | 0.0001 |
| 10 | μM | 75 ± 9 | NS | 56 ± 13 | 0.001 | 0.003 |
| 100 | μM | 76 ± 7 | NS | 52 ± 9 | 0.0001 | 0.0001 |

Platelet Aggregation in Whole Blood

Valsartan and valeryl 4-hydroxy valsartan inhibited aggregation of human platelets induced by 1 μM collagen in whole blood within the range of therapeutic activity. A dose dependent effect was observed for the both of compound, but preincubation with valeryl 4-hydroxy valsartan resulted in a significant reduction of platelet aggregability. The results of collagen-induced platelet aggregation are demonstrated by Table 2.

TABLE 2
Whole blood platelet aggregation

| | | | Valeryl 4-hydroxy valsartan | | |
|---|---|---|---|---|---|
| | | | (valeryl 4-hydroxy | | p-value |
| Variable | Valsartan (V) | | valsartan) | | V vs. |
| Drug | | p-value | | p-value | valeryl 4- |
| concen- | | vs. | | vs. | hydroxy |
| tration | | baseline | | baseline | valsartan |

Whole blood impedance platelet aggregation induced by 1 mg/ml
collagen(ohms)

| | | | | | | |
|---|---|---|---|---|---|---|
| Baseline | 29 ± 7 | | 29 ± 7 | | | |
| 10 | nM | 30 ± 8 | NS | 32 ± 8 | NS | NS |
| 100 | nM | 30 ± 7 | NS | 31 ± 9 | NS | NS |
| 1 | μM | 27 ± 7 | NS | 14 ± 6 | 0.0001 | 0.0001 |
| 10 | μM | 29 ± 6 | NS | 16 ± 8 | 0.004 | 0.001 |
| 100 | μM | 20 ± 8 | 0.02 | 17 ± 8 | 0.01 | NS |

Cartridge-Based Platelet Function Analysers (PFA-100™ and Ultegra®)

A consistent dose-dependent delay of the closure time (PFA) as a reduction of platelet aggregation units (Ultegra) was observed, indicating platelet inhibition under high shear conditions. Similar to the whole blood aggregation and epinephrine-induced aggregation, valeryl 4-hydroxy valsartan had stronger antiplatelet properties than valsartan. Table 3 demonstrates a representative experiment with cartridge-based analyzers.

TABLE 3
Cartridge-based analyzers

| | | | Valeryl 4-hydroxy valsartan | | |
|---|---|---|---|---|---|
| | | | (valeryl 4-hydroxy | | p-value |
| Variable | Valsartan (V) | | valsartan) | | V vs. |
| Drug | | p-value | | p-value | valeryl 4- |
| concen- | | vs. | | vs. | hydroxy |
| tration | | baseline | | baseline | valsartan |

PFA-100 ™ closure time (s) with the epinephrine/collagen cartridge *

| Baseline | | 201 ± 22 | | 201 ± 22 | | |
|---|---|---|---|---|---|---|
| 10 | nM | 187 ± 30 | NS | 179 ± 28 | NS | NS |
| 100 | nM | 209 ± 21 | NS | 190 ± 24 | NS | NS |
| 1 | μM | 268 ± 19 | 0.02 | 259 ± 20 | 0.03 | NS |
| 10 | μM | 278 ± 20 | 0.02 | 257 ± 25 | 0.04 | NS |
| 100 | μM | 200 ± 27 | NS | 232 ± 31 | NS | NS |

Ultegra ® (platelet activation units) Analyzer

| Baseline | | 159 ± 27 | | 159 ± 27 | | |
|---|---|---|---|---|---|---|
| 10 | nM | 164 ± 16 | NS | 177 ± 23 | NS | NS |
| 100 | nM | 160 ± 29 | NS | 181 ± 31 | NS | NS |
| 1 | μM | 129 ± 26 | NS | 134 ± 29 | NS | NS |
| 10 | μM | 130 ± 31 | NS | 119 ± 38 | NS | NS |
| 100 | μM | 152 ± 19 | NS | 168 ± 35 | NS | NS |

Whole Blood Flow Cytometry

Incubation with valsartan and valeryl 4-hydroxy valsartan slightly reduced the expression of GP IIb/IIIa (CD 41) and fibrinogen binding (PAC-1), and valeryl 4-hydroxy valsartan achieved this effect at therapeutic concentrations, whereas valsartan decreased expression of CD41 only at a high dose. Both agents significantly diminished concentration of P-selectin on platelet surface, and moderately reduced the expression of vitronectin receptors, even this effect did not have statistical power to show difference between valsartan and valeryl 4-hydroxy valsartan. The incubation with valsartan and valeryl 4-hydroxy valsartan was not associated with any effect on PECAM-1 (CD31); GP Ib (CD42) and LAMP-2 (CD107b), PETA-3 (CD151), and platelet-leukocyte microparticles (CD151+14). The results of flow cytometry are presented on Table 4.

TABLE 4
Effect of Valsartan and valeryl 4-hydroxy valsartan
on the expression of major platelet receptors

| | | | Valeryl 4-hydroxy val- | | |
|---|---|---|---|---|---|
| | | | sartan (valeryl 4- | | |
| | Valsartan (V) | | hydroxy valsartan) | | |
| | | p- | | p- | p-value |
| Variable | | value | | value | V vs. |
| Drug | | vs. | | vs. | valeryl 4- |
| concen- | | base- | | base- | hydroxy |
| tration | | line | | line | valsartan |

Expression of platelet/endothelial cell adhesion molecule-1 (PECAM-1,
CD31, log MFI)

| Baseline | | 69.2 ± 10.3 | | 69.2 ± 10.3 | | |
|---|---|---|---|---|---|---|
| 10 | nM | 71.8 ± 11.9 | NS | 74.2 ± 13.4 | NS | NS |
| 100 | nM | 70.6 ± 12.6 | NS | 69.7 ± 15.1 | NS | NS |
| 1 | μM | 74.5 ± 16.2 | NS | 70.3 ± 16.4 | NS | NS |
| 10 | μM | 68.2 ± 16.2 | NS | 73.1 ± 9.3 | NS | NS |
| 100 | μM | 70.6 ± 15.8 | NS | 71.2 ± 15.5 | NS | NS |

Expression of platelet glycoprotein IIb/IIIa antigen (GPIIb, CD41,
log MFI)

| Baseline | | 521.8 ± 98.2 | | 521.8 ± 98.2 | | |
|---|---|---|---|---|---|---|
| 10 | nM | 531.2 ± 102.5 | NS | 505.6 ± 77.8 | NS | NS |
| 100 | nM | 502.9 ± 112.7 | NS | 432.7 ± 116.8 | 0.03 | 0.04 |
| 1 | μM | 498.2 ± 94.5 | NS | 427.2 ± 114.1 | 0.02 | NS |
| 10 | μM | 506.7 ± 100.1 | NS | 515.9 ± 156.2 | NS | NS |
| 100 | μM | 455.2 ± 99.6 | 0.03 | 529.3 ± 121.8 | NS | 0.04 |

Expression of glycoprotein Ib (GPIb, CD42b, log MFI)

| Baseline | | 246.4 ± 26.7 | | 246.4 ± 26.7 | | |
|---|---|---|---|---|---|---|
| 10 | nM | 256.4 ± 31.4 | NS | 236.4 ± 18.7 | NS | NS |
| 100 | nM | 248.7 ± 20.9 | NS | 267.9 ± 39.4 | NS | NS |
| 1 | μM | 264.8 ± 18.2 | NS | 255.5 ± 24.8 | NS | NS |
| 10 | μM | 244.3 ± 46.9 | NS | 249.7 ± 41.8 | NS | NS |
| 100 | μM | 255.9 ± 19.7 | NS | 248.1 ± 19.5 | NS | NS |

Expression of platelet vitronectin receptor (CD51/CD61, log MFI)

| Baseline | | 11.2 ± 4.6 | | 11.2 ± 4.6 | | |
|---|---|---|---|---|---|---|
| 10 | nM | 11.9 ± 6.0 | NS | 12.1 ± 4.2 | NS | NS |
| 100 | nM | 12.1 ± 5.1 | NS | 8.5 ± 3.1 | 0.02 | 0.01 |
| 1 | μM | 7.8 ± 3.6 | 0.01 | 8.1 ± 3.8 | 0.02 | NS |
| 10 | μM | 7.6 ± 2.3 | 0.01 | 7.2 ± 5.6 | 0.01 | NS |
| 100 | μM | 7.3 ± 4.8 | 0.006 | 7.6 ± 3.3 | 0.01 | NS |

Expression of P-selectin (CD 62p, % of cell positivity)

| Baseline | | 9.1 ± 3.8 | | 9.1 ± 3.8 | | |
|---|---|---|---|---|---|---|
| 10 | nM | 9.8 ± 2.7 | NS | 9.3 ± 4.3 | NS | NS |
| 100 | nM | 10.3 ± 4.1 | NS | 10.2 ± 5.4 | NS | NS |
| 1 | μM | 7.2 ± 3.2 | 0.03 | 7.3 ± 3.7 | 0.03 | NS |
| 10 | μM | 7.2 ± 3.6 | 0.03 | 6.9 ± 4.2 | 0.03 | NS |
| 100 | μM | 7.8 ± 4.2 | NS | 8.9 ± 3.8 | NS | NS |

Expression of lysosome associated membrane protein -1 (LAMP-1,
CD107a, log MFI)

| Baseline | | 8.7 ± 3.2 | | 8.7 ± 3.2 | | |
|---|---|---|---|---|---|---|
| 10 | nM | 9.1 ± 3.8 | NS | 10.2 ± 3.6 | NS | NS |
| 100 | nM | 8.9 ± 2.7 | NS | 9.0 ± 4.0 | NS | NS |
| 1 | μM | 9.4 ± 3.9 | NS | 6.6 ± 2.9 | 0.04 | 0.02 |
| 10 | μM | 6.5 ± 3.2 | 0.04 | 5.8 ± 3.2 | 0.03 | NS |
| 100 | μM | 10.1 ± 4.5 | NS | 8.0 ± 3.4 | NS | NS |

Expression of lysosome associated membrane protein -2 (LAMP-2,
CD107b, log MFI)

| Baseline | | 6.6 ± 2.1 | | 6.6 ± 2.1 | | |
|---|---|---|---|---|---|---|
| 10 | nM | 7.6 ± 3.2 | NS | 7.7 ± 3.8 | NS | NS |
| 100 | nM | 7.0 ± 2.7 | NS | 7.0 ± 3.0 | NS | NS |
| 1 | μM | 6.8 ± 3.5 | NS | 6.6 ± 3.4 | NS | NS |
| 10 | μM | 7.1 ± 3.3 | NS | 7.2 ± 3.9 | NS | NS |
| 100 | μM | 7.2 ± 3.8 | NS | 7.1 ± 2.9 | NS | NS |

Expression of platelet/endothelial tetraspan antigen -3 (PETA-3,
CD151, log MFI)

| Baseline | | 88.7 ± 24.2 | | 88.7 ± 24.2 | | |
|---|---|---|---|---|---|---|
| 10 | nM | 84.1 ± 19.7 | NS | 89.4 ± 18.2 | NS | NS |
| 100 | nM | 91.5 ± 23.4 | NS | 92.7 ± 22.2 | NS | NS |
| 1 | μM | 81.6 ± 20.1 | NS | 90.5 ± 15.7 | NS | NS |
| 10 | μM | 87.9 ± 18.3 | NS | 84.2 ± 31.1 | NS | NS |
| 100 | μM | 91.7 ± 22.1 | NS | 93.4 ± 27.4 | NS | NS |

Formation of platelet-leukocyte microparticles (CD151 + CD14,
log MFI)

| Baseline | | 92.2 ± 19.3 | | 92.2 ± 19.3 | | |
|---|---|---|---|---|---|---|
| 10 | nM | 91.2 ± 20.5 | NS | 99.4 ± 29.7 | NS | NS |
| 100 | nM | 88.4 ± 21.9 | NS | 94.3 ± 18.7 | NS | NS |
| 1 | μM | 93.5 ± 23.8 | NS | 90.7 ± 20.5 | NS | NS |
| 10 | μM | 96.5 ± 18.7 | NS | 89.2 ± 18.7 | NS | NS |
| 100 | μM | 90.5 ± 19.2 | NS | 95.6 ± 19.9 | NS | NS |

Glycoprotein IIb/IIIa activity (fibrinogen binding, PAC-1, log MFI)

| Baseline | | 10.3 ± 2.8 | | 10.3 ± 2.8 | | |
|---|---|---|---|---|---|---|
| 10 | nM | 9.3 ± 2.87 | NS | 11.0 ± 4.5 | NS | NS |
| 100 | nM | 11.2 ± 3.4 | NS | 9.0 ± 3.3 | NS | NS |
| 1 | μM | 7.0 ± 2.9 | 0.03 | 6.7 ± 2.7 | 0.02 | NS |
| 10 | μM | 6.9 ± 2.7 | 0.02 | 7.5 ± 3.1 | 0.04 | NS |
| 100 | μM | 9.9 ± 3.1 | NS | 9.9 ± 3.0 | NS | NS |

The following examples illustrate the above-described invention; however, it is not intended to restrict the scope of this invention in any manner.

### EXAMPLE 1

Method of Synthesis of Valsartan Metabolite

### (S)-2{(4-Hydroxy-pentanoyl)-[2′-(1H-tetrazol-5-yl)-biphenyl-4-ylmethyl]amino}-3-methyl-butyric Acid

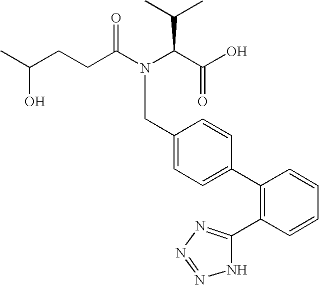

6.7 g (S3-Methyl-2-{(4-oxo-pentanoyl)-[2′-(1H-tetrazol-5-yl)-biphenyl-4-ylmethyl]-amino}-butyric acid are dissolved in 60 ml methanol and cooled to 0° C. 2.25 g sodium borohydride are added in small portions in order to keep the stirred reaction mixture below 27° C. (strong foaming). The mixture is stirred at room temperature for 1 hour, concentrated in vacuo, dissolved in methylenechloride and extracted twice with 2N aqueous hydrochloric acid. The organic phase is dried, concentrated in vacuo and the product is received by chromatography (flash column, 240 g silicagel 60, KG40-62 micrometer, using a solvent mixture of methylenechloride, methanol, conc. aqueous ammonia (30:10:1 v/v). The fractions containing the product were concentrated, dissolved in methylenechloride and extracted with 2N aqueous hydrochloric acid and dried over sodium sulfate. After concentrating the residue is dried in vacuo (60° C.) for 3 days yielding (S2-{(4-Hydroxy-pentanoyl)[2′-(1H-tetrazol-5-yl)-biphenyl-4-ylmethyl]amino}-3-methyl-butyric acid as a white foam ([α]_{D}²⁰=−58° (c=1, methanol). TLC-Rf: 0.18 (toluene/ethylacetate/methylenechloride/formic acid 16:40:40:4).

The starting material (S)-3-Methyl-2-{(4-oxo-pentanoyl)-[2′-(1H-tetrazol-5-yl)-biphenyl-4-ylmethyl]-amino}-butyric acid can be prepared as follows:

### (S)-2-{(2′-Cyano-biphenyl-4-ylmethyl)-[3-(2-methyl-[1,3]dioxolan-2-yl)-propionyl]-amino}-3-methyl-butyric Acid Benzyl Ester

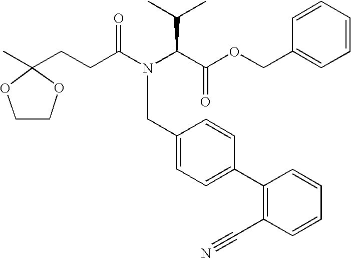

13.8 g (S)-2-[(2′-Cyano-biphenylylmethylyamino]-3-methyl-butyric acid benzyl ester Hydrochloride (described in EP 443983) are dissolved in 50 ml methylenechloride, cooled to 0° C. and treated with 23.8 ml ethyldiisopropylamine (Hünig base). To this mixture is added at 0° C. a solution of 3-(2-Methyl-[1,3]dioxolan-2-yl)-propionyl chloride, prepared from 8.9 g 3-(2-Methyl-[1,3]dioxolan-2-yl)-propionic acid (Tetrahedron 37, 307, 1981) and 10.31 ml (1-Chloro-2-methyl-propenyl)dimethyl-amine (Tetrahydron 54, 9207, 1998) in 40 ml methylenechloride. The reaction mixture is stirred at room temperature for 3-4 days, depending on the progress of the transformation. Preferably, 3-(2-Methyl-[1,3]dioxolan-2-yl)propionyl chloride is added in 3-4 portions over 2 days. The reaction mixture is concentrated in vacuo, dissolved in ethylacetate, washed with water, 1N aqueous hydrochloric acid, water, dried over sodium sulfate and concentrated in vacuo. Flash column chromatography (240 g silicagel 60, 40-63 micrometer, petroleum ether/ethylacetate 2:1 to 1:1) provides, after drying of the product in vacuo at 50 0° C., pure (S)-2-{(2′-Cyano-biphenyl-4-ylmethyl)-[3-(2-methyl-[1,3]dioxolan-2-yl)-propionyl]-amino}-3-methyl-butyric acid benzyl ester as a golden, sticky residue. TLC-Rf: 0.23 (petroleum ether/ethylacetate 2:1).

### (S)-2-[(2′-Cyano-biphenyl-4-ylmethyl)-(4-oxo-pentanoyl)-amino]-3-methyl-butyric Acid Benzyl Ester

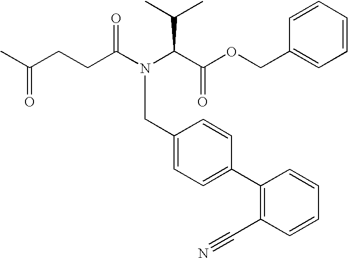

9.8 g (S)-2-{(2′-Cyano-biphenyl-4-ylmethyl)-[3-(2-methyl-[1,3]dioxolan-2-yl)-propionyl]-amino}-3-methyl-butyric acid benzyl ester are dissolved in 100 ml tetrahydrofurane and treated with 50 ml 1N aqueous hydrochloric acid. The mixture is stirred at room temperature for 6.5 hours, concentrated in vacuo and extracted with methylenechloride. The organic phase is washed with water, dried over sodium sulfate, concentrated in vacuo, evaporated and dried in vacuo at 50 0° C. for 1 hour. (S2-[(2′-Cyano-biphenyl-4-ylmethyl)-(4-oxo-pentanoyl)amino]-3-methyl-butyric acid benzyl ester is received as orange, viscous oil. THL-RF: 0.18 (petroleum ether/ethylacetate 2:1).

### (S)-3-Methyl-2-{(4-oxo-pentanoyl)-[2′-(1H-tetrazol-5-yl)-biphenyl-4-ylmethyl]-amino}-butyric Acid Benzyl Ester

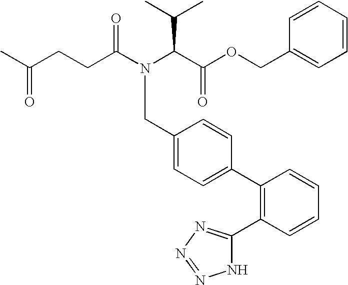

8.64 g (S)-2-[(2′-Cyano-biphenyl-4-ylmethyl)-(4-oxo-pentanoyl)-amino]-3-methyl-butyric acid benzyl ester and 12.71 g Tributyltinazid (Aldrich) in 20 ml xylene are refluxed for 28 hours. The mixture is treated with 0.5N aqueous sodium hydroxide solution, the water phase is washed with ether and ether phase is extracted once with water. The combined water phase is acidified with concentrated aqueous hydrochloric acid, extracted with methylenechloride, washed with water, suspended with active carbon, filtered, dried over sodium sulfate, concentrated and dried in vacuo. The product (S)-3-Methyl-2-{(4-oxo-pentanoyl)-[2′-(1H-tetrazol-5-yl)-biphenyl-4-ylmethyl]-amino}butyric acid benzyl ester is received as brown foam. TLC-Rf: 0.36 (toluene/methylenechloride/methanol/formic acid 40:40:40:4).

### (S)-3-Methyl-2-{(4-oxo-pentanoyl)-[2′-(1H-tetrazol-5-yl)-biphenyl-4-ylmethyl]-amino}-butyric Acid

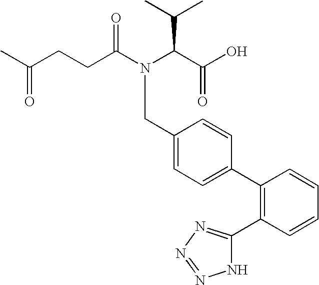

7.99 (S)-3-Methyl-2-{(4-oxo-pentanoyl)-[2-(1H-tetrazol-5-yl)-biphenyl-4-ylmethyl]-amino}-butyric acid benzyl ester in 160 ml tetrahydrofuran are hydrogenated under normal pressure at room temperature in the presence of 1.5 g palladium on carbon (10%) until saturation is achieved. The mixture is filtered and concentrated in vacuo providing (S)-3-Methyl-2-{(4-oxo-pentanoyl)-[2′-(1H-tetrazol-5-yl)biphenyl-4-ylmethyl]-amino}butyric acid as an almost white foam. TLC-Rf: 0.1 (toluene/methylenechloride/methanol/formic acid 40:40:40:4).

Although the present invention has been described in considerable detail with reference to certain preferred versions thereof, other versions are possible without departing from the spirit and scope of the preferred versions contained herein.

All publications and patents mentioned herein are incorporate by reference in their entirety as if set forth in full herein.

1. A method for inhibiting platelet aggregation comprising administering a therapeutically effective amount of the metabolite valeryl 4-hydroxy valsartan or a pharmaceutically acceptable salt thereof, optionally in the presence of a pharmaceutically acceptable carrier, to a patient in need thereof.

2. A method of for the prevention, delay of progression or treatment of conditions mediated by platelet aggregation comprising administering a therapeutically effective amount of the metabolite valeryl 4-hydroxy valsartan or a pharmaceutically acceptable salt thereof, to a patient in need thereof.

3. The method of claim 2 wherein the condition mediated by platelet aggregation is acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.

4. A pharmaceutical composition comprising a therapeutically effective amount of the metabolite valeryl 4-hydroxy valsartan or a pharmaceutically acceptable salt thereof and a pharmaceutically acceptable carrier.

5. Use of the metabolite valeryl 4-hydroxy valsartan or a pharmaceutically acceptable salt thereof for the manufacture of a medicament for the prevention, delay of progression or treatment of a disease and disorder mediated by platelet aggregation, in particular, acute myocardial infarction, ischemic stroke, angina pectoris, acute coronary syndromes, TIA (transient ischemic attacks, or acute cerebrovascular syndromes), heart failure, chest pain of ischemic etiology, syndrome X, thromboembolism, pulmonary hypertension, diabetes mellitus, peripheral vascular disease, deep vein thrombosis, arterial thrombosis of any vessel, catheter thrombotic occlusion or reocclusion.
